(19) United States
(12) Patent Application Publication
(10) Pub. No.: US 2024/0086535 A1
(43) Pub. Date: Mar. 14, 2024

(54) MULTI-MICRO-ORGAN SYSTEM DATA PROCESSING SYSTEM BASED ON FIELD PROGRAMMABLE GATE ARRAY

(71) Applicants: Yulin CAO, Jiyuan (CN); Tangyi Holding (Shenzhen) Co., Ltd., Shenzhen (CN)

(72) Inventor: Yulin CAO, Beijing (CN)

(73) Assignees: Yulin CAO, Jiyuan (CN); Tangyi Holding (Shenzhen) Co., Ltd., Shenzhen (CN)

(21) Appl. No.: 18/086,695

(22) Filed: Dec. 22, 2022

(30) Foreign Application Priority Data
Sep. 14, 2022 (CN) 202211114434.5

(51) Int. Cl. G06F 21/56; G06F 12/0864

(52) U.S. Cl. CPC G06F 21/566; G06F 12/0864; G06F 21/568

(57) ABSTRACT

A multi-micro-organ system data processing system based on a field programmable gate array includes a data collection module, a programmable processing terminal, a server and a display terminal, the data collection module is connected to the programmable processing terminal by means of a signal line, the programmable processing terminal is connected to the server, the server is connected to the display terminal, and the programmable processing terminal includes a data processing chip, a file management module, a cache module, a high-speed transmission module, a safety protection module and a fault alarm module. According to the disclosure, flow data, temperature data, CO₂ concentration data, cell activity data, functional index data and electroencephalogram signals can be synchronously collected, quickly processed, and then cached and transmitted at a high speed, so as to obviously improve data processing efficiency.

### CROSS REFERENCE TO THE RELATED APPLICATIONS

This application is based upon and claims priority to Chinese Patent Application No. 202211114434.5, filed on Sep. 14, 2022, the entire contents of which are incorporated herein by reference.

### TECHNICAL FIELD

The disclosure relates to the technical field of multi-micro-organ system data processing, and particularly to a multi-micro-organ system data processing system based on a field programmable gate array.

### BACKGROUND

Aiming at treating and preventing physiological diseases and improving physiological health of human bodies, medicine not only is a discipline for treating various diseases or pathological changes of life, and a major for promoting recovery of patients, but also is a science for dealing with the problems related to desirable physiological states of human bodies in the definition of human health. Medicine means treatment to diseases and recovery of effective functions of bodies in a narrow sense, while further includes chinese traditional health preservation and western nutrition derived from it in a broad sense.

In the process of medical research, research on multiple micro-organs is often involved, during which data is required to be collected. Moreover, the data is required to be processed by a data processing system. However, the existing data processing system cannot synchronously collect the data, and cannot cache and transmit the data at a high speed after collecting the data, so as to affect the data processing efficiency. Therefore, the system has room for improvement.

### SUMMARY

The objective of the disclosure is to provide a multi-micro-organ system data processing system based on a field programmable gate array, so as to solve the defects in the prior art. The disclosure has the advantages that flow data, temperature data, CO₂ concentration data, cell activity data, functional index data and electroencephalogram signals may be synchronously collected, quickly processed, and then cached and transmitted at a high speed, so as to obviously improve data processing efficiency.

In order to realize the above objective, the disclosure employs the technical solution as follows:
a multi-micro-organ system data processing system based on a field programmable gate array includes a data collection module, a programmable processing terminal, a server and a display terminal, the data collection module is connected to the programmable processing terminal by means of a signal line, the programmable processing terminal is connected to the server, the server is connected to the display terminal, the programmable processing terminal includes a data processing chip, a file management module, a cache module, a high-speed transmission module, a safety protection module and a fault alarm module, and the data processing chip is connected to the file management module, the cache module, the high-speed transmission module, the safety protection module and the fault alarm module separately by means of signal lines.

The disclosure is further configured as follows: the data collection module includes a flow collection unit, a temperature collection unit and a CO₂ concentration collection unit, the flow collection unit is composed of several flow sensors for synchronously collecting flow data, the temperature collection unit is composed of several temperature sensors for synchronously collecting temperature data, and the CO₂ concentration collection unit is composed of several CO₂ concentration sensors for synchronously collecting CO₂ concentration data.

The disclosure is further configured as follows: the data collection module further includes a cell activity collection unit, a functional index collection unit and an electroencephalogram signal collection unit, the cell activity collection unit is composed of several cell activity sensors for collecting cell activity data, the functional index collection unit is composed of several functional index sensors for collecting functional index data, and the electroencephalogram signal collection unit is composed of several electroencephalogram signal sensors for collecting electroencephalogram signal data.

The disclosure is further configured as follows: the file management module includes a file classification unit, a file retrieval unit and a file modification unit, the file classification unit is used for effectively classifying system files, the file retrieval unit is used for quickly retrieving system files to be found, and the file modification unit is used for conveniently modifying data in the system files.

The disclosure is further configured as follows: the safety protection module includes a firewall presetting unit, a virus finding unit and a virus killing and removing unit, the firewall presetting unit is used for presetting a firewall for a system, so as to prevent intrusion of hackers, the virus finding unit is used for quickly locating a file infected with a virus, and the virus killing and removing unit is used for killing and removing the virus after infection, so as to improve safety of the system.

The disclosure is further configured as follows: the high-speed transmission module includes a wired transmission unit and a wireless transmission unit, the wired transmission unit carries out connection by means of a high definition multimedia interface (HDMI) cable, and the wireless transmission unit carries out connection by means of a wireless local area network.

The disclosure is further configured as follows: the cache module is at least one of a direct-mapped cache, a set associative cache and a fully associative cache, and preferably a fully associative cache.

The disclosure is further configured as follows: the fault alarm module includes an audible alarm unit and a pop-up alarm unit, the audible alarm unit is used for raising the voice alarm when the system is abnormal, and the pop-up alarm unit is used for raising the pop-up alarm when the system is abnormal.

The disclosure is further configured as follows: the data processing chip is a field programmable gate array chip, and the field programmable gate array chip may simultaneously process data synchronously collected.

The disclosure is further configured as follows: the display terminal includes a mobile display terminal and a non-mobile display terminal, the mobile display terminal includes a mobile phone, a tablet computer and a notebook computer, and the non-mobile display terminal is a liquid crystal display.

The disclosure has the beneficial effects: according to the multi-micro-organ system data processing system based on a field programmable gate array, by means of the data collection module, flow data, temperature data, CO₂ concentration data, cell activity data, functional index data and electroencephalogram signals may be synchronously collected, quickly processed, and then cached and transmitted at a high speed, so as to obviously improve data processing efficiency.

### BRIEF DESCRIPTION OF THE DRAWINGS

FIG. 1 is a schematic diagram of an overall structure of a multi-micro-organ system data processing system based on a field programmable gate array provided in the disclosure;

FIG. 2 is a schematic structural diagram of a data collection module of a multi-micro-organ system data processing system based on a field programmable gate array provided in the disclosure;

FIG. 3 is a schematic structural diagram of a file management module of a multi-micro-organ system data processing system based on a field programmable gate array provided in the disclosure; and

FIG. 4 is a schematic structural diagram of a safety protection module of a multi-micro-organ system data processing system based on a field programmable gate array provided in the disclosure.

### DETAILED DESCRIPTION OF THE EMBODIMENTS

The technical solution of the patent will be further described in detail below in combination with the specific embodiments.

Examples of the patent will be described in detail below, and instances of the examples are shown in accompanying drawings, throughout which identical or similar reference numerals denote identical or similar elements or elements having identical or similar functions. The examples described below with reference to the accompanying drawings are exemplary and are merely used to explain the patent, but cannot be construed as limiting the patent.

In the description of the patent, it is to be understood that orientation or positional relations indicated by the terms “center”, “upper”, “lower”, “front”, “rear”, “left”, “right”, “vertical”, “horizontal”, “top”, “bottom”, “inside”, “outside”, etc. are based on orientation or positional relations shown in the accompanying drawings, are merely for facilitating the description of the patent and simplifying the description, rather than indicating or implying that an apparatus or element referred to must have a particular orientation or be constructed and operated in a particular orientation, and therefore will not be construed as limiting the patent.

In the description of the patent, it should be noted that unless explicitly specified and defined otherwise, the terms “mount”, “connected”, “connect”, and “arranged” should be understood broadly, for example, they can mean fixed connection and arrangement, detachable connection and arrangement, or integral connection and arrangement. For those of ordinary skill in the art, the specific meanings of the above terms in the patent could be understood according to specific circumstances.

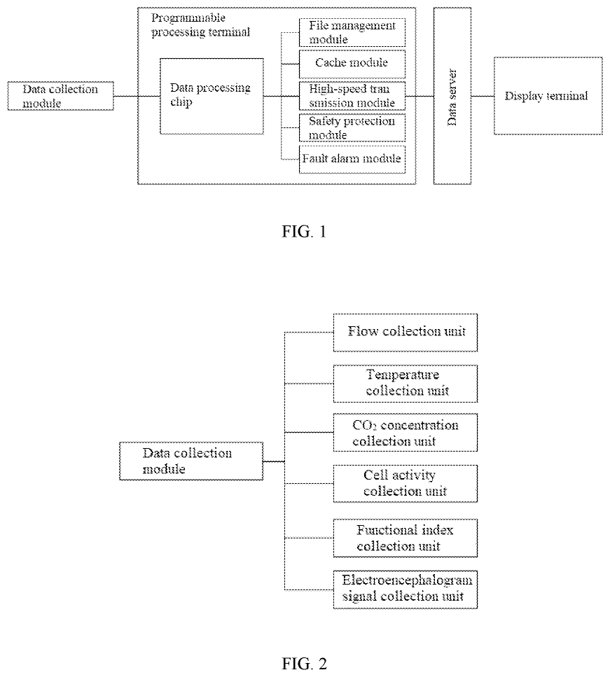

With reference to FIG. 1, a multi-micro-organ system data processing system based on a field programmable gate array includes a data collection module, a programmable processing terminal, a server and a display terminal, the data collection module is connected to the programmable processing terminal by means of a signal line, the programmable processing terminal is connected to the server, the server is connected to the display terminal, the programmable processing terminal includes a data processing chip, a file management module, a cache module, a high-speed transmission module, a safety protection module and a fault alarm module, and the data processing chip is connected to the file management module, the cache module, the high-speed transmission module, the safety protection module and the fault alarm module separately by means of signal lines.

With reference to FIG. 2, the data collection module includes a flow collection unit, a temperature collection unit and a CO₂ concentration collection unit, the flow collection unit is composed of several flow sensors for synchronously collecting flow data, the temperature collection unit is composed of several temperature sensors for synchronously collecting temperature data, and the CO₂ concentration collection unit is composed of several CO₂ concentration sensors for synchronously collecting CO₂ concentration data. The data collection module further includes a cell activity collection unit, a functional index collection unit and an electroencephalogram signal collection unit, the cell activity collection unit is composed of several cell activity sensors for collecting cell activity data, the functional index collection unit is composed of several functional index sensors for collecting functional index data, and the electroencephalogram signal collection unit is composed of several electroencephalogram signal sensors for collecting electroencephalogram signal data.

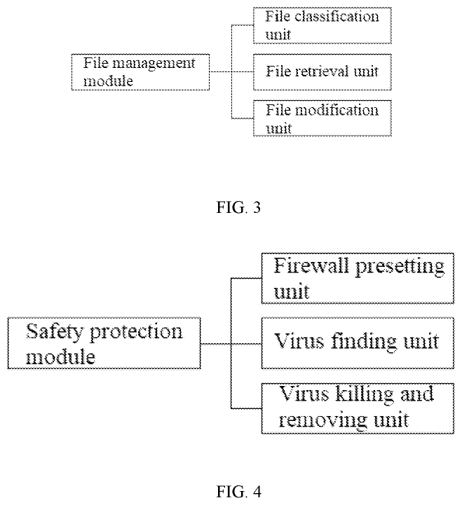

With reference to FIG. 3, the file management module includes a file classification unit, a file retrieval unit and a file modification unit, the file classification unit is used for effectively classifying system files, the file retrieval unit is used for quickly retrieving system files to be found, and the file modification unit is used for conveniently modifying data in the system files.

With reference to FIG. 4, the safety protection module includes a firewall presetting unit, a virus finding unit and a virus killing and removing unit, the firewall presetting unit is used for presetting a firewall for a system, so as to prevent intrusion of hackers, the virus finding unit is used for quickly locating a file infected with a virus, and the virus killing and removing unit is used for killing and removing the virus after infection, so as to improve safety of the system.

In the example, the high-speed transmission module includes a wired transmission unit and a wireless transmission unit, the wired transmission unit carries out connection by means of a high definition multimedia interface (HDMI) cable, and the wireless transmission unit carries out connection by means of a wireless local area network. The cache module is at least one of a direct-mapped cache, a set associative cache and a fully associative cache, and preferably a fully associative cache. The fault alarm module includes an audible alarm unit and a pop-up alarm unit, the audible alarm unit is used for raising the voice alarm when the system is abnormal, and the pop-up alarm unit is used for raising the pop-up alarm when the system is abnormal. The data processing chip is a field programmable gate array chip, and the field programmable gate array chip may simultaneously process data synchronously collected. The display terminal includes a mobile display terminal and a non-mobile display terminal, the mobile display terminal includes a mobile phone, a tablet computer and a notebook computer, and the non-mobile display terminal is a liquid crystal display.

What is mentioned above is merely optimal specific embodiments of the disclosure, but is not intended to limit the scope of protection of the disclosure. Any equivalent substitutions or changes made by those skilled in the art according to the technical solution of the disclosure and the inventive concept thereof within the scope of technology disclosed in the disclosure should fall within the scope of protection of the disclosure.

What is claimed is:

1. A multi-micro-organ system data processing system based on a field programmable gate array, comprising a data collection module, a programmable processing terminal, a server, and a display terminal,
wherein the data collection module is connected to the programmable processing terminal by a signal line, the programmable processing terminal is connected to the server, the server is connected to the display terminal,
the programmable processing terminal comprises a data processing chip, a file management module, a cache module, a high-speed transmission module, a safety protection module, and a fault alarm module, and the data processing chip is connected to the file management module, the cache module, the high-speed transmission module, the safety protection module, and the fault alarm module separately by the signal line.

2. The multi-micro-organ system data processing system based on the field programmable gate array according to claim 1, wherein the data collection module comprises a flow collection unit, a temperature collection unit, and a CO₂ concentration collection unit, the flow collection unit is composed of a plurality of flow sensors for synchronously collecting a flow data, the temperature collection unit is composed of a plurality of temperature sensors for synchronously collecting a temperature data, and the CO₂ concentration collection unit is composed of a plurality of CO₂ concentration sensors for synchronously collecting a CO₂ concentration data.

3. The multi-micro-organ system data processing system based on the field programmable gate array according to claim 2, wherein the data collection module further comprises a cell activity collection unit, a functional index collection unit, and an electroencephalogram signal collection unit, the cell activity collection unit is composed of a plurality of cell activity sensors for collecting a cell activity data, the functional index collection unit is composed of a plurality of functional index sensors for collecting a functional index data, and the electroencephalogram signal collection unit is composed of a plurality of electroencephalogram signal sensors for collecting an electroencephalogram signal data.

4. The multi-micro-organ system data processing system based on the field programmable gate array according to claim 1, wherein the file management module comprises a file classification unit, a file retrieval unit and a file modification unit, the file classification unit is used for effectively classifying system files, the file retrieval unit is used for quickly retrieving system files to be found, and the file modification unit is used for conveniently modifying a data in the system files.

5. The multi-micro-organ system data processing system based on the field programmable gate array according to claim 4, wherein the safety protection module comprises a firewall presetting unit, a virus finding unit, and a virus killing and removing unit, the firewall presetting unit is used for presetting a firewall for the multi-micro-organ system data processing system based on the field programmable gate array, so as to prevent an intrusion of hackers, the virus finding unit is used for quickly locating a file infected with a virus, and the virus killing and removing unit is used for killing and removing the virus after an infection, so as to improve safety of the multi-micro-organ system data processing system based on the field programmable gate array.

6. The multi-micro-organ system data processing system based on the field programmable gate array according to claim 5, wherein the high-speed transmission module comprises a wired transmission unit and a wireless transmission unit, the wired transmission unit carries out a connection by a high definition multimedia interface (HDMI) cable, and the wireless transmission unit carries out the connection by a wireless local area network.

7. The multi-micro-organ system data processing system based on the field programmable gate array according to claim 6, wherein the cache module is at least one of a direct-mapped cache, a set associative cache, and a fully associative cache.

8. The multi-micro-organ system data processing system based on the field programmable gate array according to claim 7, wherein the fault alarm module comprises an audible alarm unit and a pop-up alarm unit, the audible alarm unit is used for raising a voice alarm when the multi-micro-organ system data processing system based on the field programmable gate array is abnormal, and the pop-up alarm unit is used for raising a pop-up alarm when the multi-micro-organ system data processing system based on the field programmable gate array is abnormal.

9. The multi-micro-organ system data processing system based on the field programmable gate array according to claim 1, wherein the data processing chip is a field programmable gate array chip, and the field programmable gate array chip is allowed to simultaneously process data synchronously collected.

10. The multi-micro-organ system data processing system based on the field programmable gate array according to claim 1, wherein the display terminal comprises a mobile display terminal and a non-mobile display terminal, the mobile display terminal comprises a mobile phone, a tablet computer, and a notebook computer, and the non-mobile display terminal is a liquid crystal display.

11. The multi-micro-organ system data processing system based on the field programmable gate array according to claim 7, wherein the cache module is the fully associative cache.
